(19) United States
(12) Patent Application Publication
(10) Pub. No.: US 2013/0071403 A1
(43) Pub. Date: Mar. 21, 2013

(54) SYNERGISTIC ANTI-TUMOR EFFICACY USING ALLOANTIGEN COMBINATION IMMUNOTHERAPY

(71) Applicant: Vical Incorporated, San Diego, CA (US)

(72) Inventors: Alain P. ROLLAND, San Diego, CA (US); John Doukas, Encinitas, CA (US); Dmitri Kharkevitch, San Diego, CA (US)

(73) Assignee: Vical Incorporated, San Diego, CA (US)

(21) Appl. No.: 13/622,210

(22) Filed: Sep. 18, 2012

Related U.S. Application Data
(60) Provisional application No. 61/536,999, filed on Sep. 20, 2011.

(51) Int. Cl. A61K 39/395; A61P 35/04; A61P 35/00

(52) U.S. Cl. 424/142.1; 424/172.1

(57) ABSTRACT

The present disclosure provides combinations of immunotherapeutics and methods for treating medical conditions that are characterized by the lack of an effective immune response, for example as would result following a down-regulation of MHC class I, such as in cancer. The immunotherapeutic compositions of the invention, which can be used to treat the medical conditions, include one or more immunostimulatory antibodies or molecules having specificity for CTLA-4, PD-1, PD-L1, PD-L2, CD40, OX40, CD137, GITR, ILT2, or ILT3, or ligands for these molecules (e.g., an isolated fully-human monoclonal antibody) in association with one or more alloantigens, such as, vector(s) capable of expressing protein(s) or peptide(s) that stimulate T-cell immunity against tissues or cells, formulated in a pharmaceutically acceptable carrier. The proteins or peptides may comprise class I major histocompatibility complex (MHC) antigens, β2-microglobulins, or cytokines. The MHC antigen may be foreign to the subject. The MHC antigen may be HLA-B7.

### RELATED APPLICATIONS

This application claims benefit of priority under 35 U.S.C. §119(e) from U.S. Provisional Application Ser. No. 61/536,999, filed Sep. 20, 2011, currently pending, the entire content of which is incorporated by reference as if fully set forth.

### BACKGROUND OF THE INVENTION

1. Field of the Invention

The present invention relates to therapeutic compositions and methods for the treatment of cancer. More particularly the invention pertains to a combination use of therapeutic compositions and methods for the treatment of melanoma.

2. Description of the State of Art

A variety of genetic abnormalities arise in human cancer that contribute to neoplastic transformation and malignancy. Instability of the genome generates mutations that alter cell proliferation, angiogenesis, metastasis, and tumor immunogenicity. Despite a better understanding of the molecular basis of cancer, many malignancies remain resistant to traditional forms of treatment.

Immunotherapy has shown promise as a primary approach to the treatment of malignancy. Indeed, specific cancers, such as melanoma or renal cell carcinoma, are relatively more responsive to modulation of immune function, possibly because the immune system can be induced to recognize mutant gene products in these cells.

In some instances, the immune system appears to contribute to the surveillance and destruction of neoplastic cells, by mobilization of either cellular or humoral immune effectors. Cellular mediators of anti-tumor activity include MHC-restricted cytotoxic T cells, natural killer (NK) cells (R. K. Oldham, Canc. Metast. Rev. 2, 323 (1983); R. B. Herberman, Concepts Immunopathol. 1, 96 (1985)) and lymphokine-activated killer (LAK) cells (S. A. Rosenberg, Immunol. Today 9, 58 (1988)). Cytolytic T cells which infiltrate tumors have been isolated and characterized (I. Yron, et al., J. Immunol. 125, 238 (1980)). These tumor infiltrating lymphocytes (TIL) selectively lyse cells of the tumor from which they were derived (P. J. Spiess, et al., J. Natl. Canc. Inst. 79, 1067; S. A. Rosenberg, et al., Science 223, 1318 (1986)). Macrophages can also kill neoplastic cells through antibody-dependent mechanisms (J. Marcelletti and P. Furmanski, J. Immunol. 120, 1 (1978); P. Ralph, et al., J. Exp. Med. 167, 712 (1988)), or by activation induced by substances such as bacillus Calmette-Guerin (BCG) (P. Alexander, Natl. Cancer Inst. Monogr. 39, 127 (1973)).

Cytokines can also participate in the anti-tumor response, either by a direct action on cell growth or by activating cellular immunity. The cytostatic effects of tumor necrosis factor-α(TNF-α) (L. J. Old, Science 230, 630 (1985)) and lymphotoxin (M. B. Powell, et al., Lymphokin Res. 4, 13 (1985)) can result in neoplastic cell death. Interferon-γ (IFN-γ) markedly increases class I MHC cell surface expression (P. Lindahl, et al., Proc. Natl. Acad. Sci. USA 70, 2785 (1973); P. Lindahl, et al., Proc. Natl. Acad. Sci. USA 73, 1284 (1976)) and synergizes with TNF-α in producing this effect (L. J. Old, Nature 326, 330 (1987)). Colony stimulating factors such as G-CSF and GM-CSF activate neutrophils and macrophages to lyse tumor cells directly (S. C. Clark and R. Kamen, Science 236, 1229 (1987)), and interleukin-2 (IL-2) activates Leu-19+ NK cells to generate lymphokine activated killer cells (LAK) capable of lysing autologous, syngeneic or allogeneic tumor cells but not normal cells (S. A. Rosenberg, Immunol. Today 9, 58 (1988); M. T. Lotze, et al., Cancer Res. 41, 4420 (1981); C. S. Johnson, et al., Cancer Res. 50, 5682 (1990)). The LAK cells lyse tumor cells without preimmunization or MHC restriction (J. H. Phillips and L. L. Lanier, J. Exp. Med. 164, 814 (1986)). Interleukin-4 (IL-4) also generates LAK cells and acts synergistically with IL-2 in the generation of tumor specific killers cells (J. J. Mule, et al., J. Immunol. 142, 726 (1989)).

Since most malignancies arise in immunocompetent hosts, it is likely that tumor cells have evolved mechanisms to escape host defenses, perhaps through evolution of successively less immunogenic clones (G. Klein and E. Klein, Proc. Natl. Acad. Sci. USA 74, 2121 (1977)). Several studies suggest that reduced expression of MHC molecules may provide a mechanism to escape detection by the immune system. Normally, the class I MHC glycoprotein is highly expressed on a wide variety of tissues and, in association with β2-microglobulin, presents endogenously synthesized peptide fragments to CD8 positive T cells through specific interactions with the CD8/T-cell receptor complex (P. J. Bjorkman and P. Parham, Ann. Rev. Biochem. 59, 253 (1990). Deficient expression of class I MHC molecules could limit the ability of tumor cells to present antigens to cytotoxic T cells. Freshly isolated cells from naturally occurring tumors frequently lack class I MHC antigen completely or show decreased expression (C. A. Holden, et al., J. Am. Acad. Dermatol. 9, 867 (1983); N. Isakov, et al., J. Natl. Canc. Inst. 71, 139 (1983); W. Schmidt, et al., Immunogen. 14, 323 (1981); K. Funa, et al., Lab Invest. 55, 185 (1986); L. A. Lampson, et al., J. Immunol. 130, 2471 (1983)). Reduced class I MHC expression could also facilitate growth of these tumors when transplanted into syngeneic recipients. Several tumor cell lines which exhibit low levels of class I MHC proteins become less oncogenic when expression vectors encoding the relevant class I MHC antigen are introduced into them (K. Tanaka, et al., Science 228, 26 (1985); K. Hui, et al., Nature 311, 750 (1984); R. Wallich, et al., Nature 315, 301 (1985); H-G. Ljunggren and K. Karre, J. Immunogenet. 13, 141 (1986); G. J. Hammerling, et al., J. Immunogenet. 13, 153 (1986)). In some experiments, tumor cells which express a class I MHC gene confer immunity in naive recipients against the parental tumor (K. Hui and F. Grosveld, H. Festenstein, Nature 311, 750 (1984); R. Wallich, et al., Nature 315, 301 (1985)).

The immune response to tumor cells can be stimulated by systemic administration of IL-2 (M. T. Lotze, et al, J. Immunol. 135, 2865 (1985)), or IL-2 with LAK cells (S. A. Rosenberg, et al., N. Eng. J. Med. 316, 889 (1987); C. S. Johnson, et al., Leukemia 3, 91 (1989)) and the ability of interferon-α to prolong the disease-free survival of patients in the adjuvant setting. (J. M. Kirkwood, et al., J. Clin Oncol. 14(1):7-17 (1996)). Recently, several studies have examined the tumor suppressive effect of lymphokine production by genetically altered tumor cells. The introduction of tumor cells transfected with an IL-2 expression vector into syngeneic mice stimulated an MHC class I restricted cytolytic T lymphocyte response which protected against subsequent rechallenge with the parental tumor cell line (E. R. Fearon, et al., Cell 60, 397 (1990)). These studies demonstrate that cytokines, expressed at high local concentrations, are effective anti-tumor agents.

### Paths to Improved Immunotherapies

As discussed previously, it is now generally accepted that immunotherapy has a role in the treatment of cancers, such as but not limited to, advanced melanoma. Research has therefore been focused on the development of immunotherapies, such as gene therapy and immunostimulatory antibodies, that may benefit a larger number of patients.

### Gene Therapy

Early studies focused on the demonstration that specific reporter genes could be expressed in vivo (E. G. Nabel, et al., Science 249, 1285 (1990); E. G. Nabel, et al., Science 244, 1342 (1989)). Subsequent studies were designed to determine whether specific biologic responses could be induced at sites of recombinant gene transfer. To address this question, a highly immunogenic molecule, a foreign major histocompatibility complex (MHC), was used to elicit an immune response in the iliofemoral artery using a porcine model. The human HLA-B7 gene was introduced using direct gene transfer with a retroviral vector or DNA liposome complex (E. G. Nabel, et al., Proc. Natl. Acad. Sci. USA 89, 5157 (1992)). With either delivery system, expression of the recombinant HLA-B7 gene product could be demonstrated at specific sites within the vessel wall. More importantly, the expression of this foreign histocompatibility antigen induced an immunologic response at the sites of genetic modification. This response included a granulomatous mononuclear cell infiltrate beginning 10 days after introduction of the recombinant gene. This response resolved by 75 days after gene transfer; however, a specific cytolytic T cell response against the HLA-B7 molecule was persistent. This study demonstrated that a specific immunologic response could be induced by the introduction of a foreign recombinant gene at a specific site in vivo. Moreover, this study provided one of the first indications that direct gene transfer of specific recombinant genes could elicit an immune response to the product of that gene in vivo (E. G. Nabel, et al., Proc. Natl. Acad. Sci. USA 89, 5157 (1992)).

These early studies demonstrated the proof of concept that eventually led to the recent enrollment completion of a phase III clinical trial of a DNA-based immunotherapy (Allovectin®) designed to overcome the down-regulation of MHC class I and as a result, induce anti-tumor responses following intratumoral (i.t.) delivery. Composed of a bicistronic plasmid (encoding HLA-B7 heavy chain and β2-microglobulin) formulated with a cationic lipid-based system (DMRIE-DOPE), Allovectin®, while not wishing to be bound by any particular theory, is believed to act through multiple mechanisms of action (MOA): (i) induction of anti-tumor T cells following tumor cell expression of the alloantigen HLA-B7 in HLA-B7 negative patients, (ii) induction of anti-tumor T cells following restoration of tumor MHC class I expression and antigen presentation, and (iii) recruitment of immune cells into tumors through the pro-inflammatory action of DNA-lipid complexes. Generation of anti-tumor T cells drives the destruction of not only those tumor sites directly injected with Allovectin®, but also distal lesions and metastases. In a recent Phase II trial in humans, no toxicity of this form of treatment was observed. It is an object of the present invention to optimize this gene therapy approach.

### Immunostimulatory Antibody Therapy

Another area of recent research interest is immunologic checkpoint blockade; the best-known therapeutics in this new field are immunostimulatory antibodies such as those that block cytotoxic T-lymphocyte antigen 4 (CTLA-4). CTLA-4 (also known as cluster of differentiation or CD152) is best characterized as a ‘brake’ that binds to costimulatory molecules on antigen-presenting cells, preventing their interaction with CD28 on T cells and also generating an overtly inhibitory signal constraining further T cell activation. CTLA-4 acts to prevent hyperstimulation of T cells that could lead to harmful autoimmunity or activation-induced cell death of T cells. The functional role of CTLA-4 is best demonstrated by the lethal autoimmunity observed in CTLA-4 knockout mice. However, temporary inhibition of CTLA-4 has been hypothesized to allow for more robust T cell activation. The first anti-CTLA-4 antibody was made in an attempt to provide a limited release of this immunologic braking mechanism, in the hope of permitting the immune system to recognize targets on tumor cells more effectively. Initial laboratory experiments demonstrated that anti-CTLA-4 antibodies used as monotherapies could indeed mediate rejection of some mouse tumors. For the well-known B16 mouse melanoma, anti-CTLA-4 therapy could provide long-term protection from tumor challenge, but only when combined with a GM-CSF-secreting tumor cell vaccine (A. van Elsas, et al., J. Exp. Med. 190, 355 (1999)). Improved anti-tumor responses were seen when programmed death 1 protein (PD-1 or CD279) and/or PD-1 ligand 1 (PD-L1 or CD274), two additional T cell negative regulators, were targeted for blockade by monoclonal antibodies (M. A. Curran, et al., Proc. Natl. Acad. Sci. USA 107, 4275 (2010)). These last results have encouraged the clinical development of anti-PD-1 and anti-PD-L1 antibodies as immunotherapies for solid tumors, with encouraging results for both (S. L. Topalian, et al., New Engl. J. Med. 366, 2443 (2012); J. R. Brahmer, et al., New Engl. J. Med. 366, 2455 (2012)). Other molecules also represent promising targets for immunostimulatory antibody therapy, as either their blockade or engagement by antibodies would be expected to reduce T cell suppression and/or activate T cells and/or other immune cells. These molecular targets include CD40, OX40 (CD134), the tumor necrosis factor receptor superfamily members 9 (CD137) and 18 (also known as glucocorticoid-induced tumor necrosis factor receptor-related protein or GITR), and the immunoglobulin-like transcript (ILT) family members ILT2 and ILT3.

Human monoclonal antibodies designed to block T cell regulators have been used in clinical trials in melanoma. For example, ipilimumab is a fully human anti-CTLA-4 monoclonal antibody developed by Medarex and Bristol-Myers Squibb which recently received FDA approval for use in melanoma. Clinical trials for ipilimumab have also revealed a unique panel of mechanism-based immune-related adverse events. The vast majority of the immune-related adverse events are low-grade pruritus and diarrhea, while some cases of more serious colitis, hepatitis and hypophysitis also have been described.

Even more intriguing is the description of new lesions occurring in the context of response in baseline tumors. Such patients would be categorized as having progression of disease by standard response criteria. However, at least a subset of such patients will have eventual regression of the new lesions, albeit later than the target lesions. To date, the best hypothesis for these varying delayed responses is that the immune system may require time to sculpt responses to different tumors with potentially different antigens. There is also inherent biologic variation in the threshold for induction of an immune response.

Therefore, there is need to provide better immunotherapies, which can elicit a robust immune response that is safe, cell or antigen-specific and effective to prevent and/or treat diseases amenable to treatment by elicitation of an immune response, such as cancer.

### SUMMARY OF THE INVENTION

The present invention provides an immunotherapeutic composition including (a) one or more binding components, in association with (b) one or more immunostimulatory therapeutic nucleic acid(s) and, optionally, a pharmaceutically acceptable carrier.

The present invention further provides an immunotherapeutic composition including (a) one or more binding components, in association with (b) one or more immunostimulatory therapeutic nucleic acid(s) capable of expressing protein(s) or peptide(s) that stimulate T-cell immunity against tissues or cells and, optionally, a pharmaceutically acceptable carrier. In some embodiments of the immunotherapeutic compositions, the one or more binding component(s) is a molecule that binds with specificity to CTLA-4, PD-1, PD-L1, PD-L2, CD40, OX40, CD137, GITR, ILT2, or ILT3, or a molecule that binds with specificity to a ligand of CTLA-4, PD-1, PD-L1, PD-L2, CD40, OX40, CD137, GITR, ILT2, or ILT3. In some embodiments, the molecule binds with specificity to a ligand of molecule that binds with specificity to a ligand of CTLA-4, PD-1, CD40, OX40, CD137, GITR, ILT2, or ILT3. Such molecules that bind with specificity may be an organic molecule, a nucleic acid molecule, or a polypeptide.

The present invention further provides an immunotherapeutic composition including (a) one or more binding components, wherein the one or more binding component is an antibody having specificity to CTLA-4, PD-1, PD-L1, PD-L2, CD40, OX40, CD137, GITR, ILT2, or ILT3, or an antibody having specificity to a ligand of CTLA-4, PD-1, PD-L1, PD-L2, CD40, OX40, CD137, GITR, ILT2, or ILT3, in association with (b) one or more immunostimulatory therapeutic nucleic acid molecule(s) capable of expressing protein(s) or peptide(s) that stimulate T-cell immunity against tissues or cells and, optionally, a pharmaceutically acceptable carrier. In some embodiments, the antibody is an isolated fully-human monoclonal antibody. In particular embodiments, the antibody binds with specificity to CTLA-4, PD-1, or PD-L1. In preferred embodiments, the antibody binds with specificity to CTLA-4. In some embodiments the human monoclonal antibody is ipilimumab, BMS-936558, BMS-936559, BMS-663513 or urelumab, CT-011 or pidilizumab, MK-3475, MPDL3280A or RG7446, CP-870,893, TRX518, or TRX385.

In further embodiments of the above immunotherapeutic compositions, the protein(s) encoded by the immunostimulatory therapeutic nucleic acid molecule(s) may be a class I major histocompatibility complex (MHC) antigen, a β2-microglobulin, or a cytokines The MHC antigen may be foreign to the subject to which the therapeutic composition is administered. The MHC antigen may be HLA-B7. The peptide(s) may compromise antigenic determinants of proteins expressed on tumors (tumor antigens) or proteins foreign to the host to which the therapeutic composition is administered. In particular embodiments, the immunostimulatory nucleic acid molecule encodes HLA-B7 heavy chain and β2-microglobulin. In some embodiments the nucleic acid molecule is a plasmid encoding HLA-B7 heavy chain and β2-microglobulin and is formulated with DMRIE-DOPE. In particular embodiments, the plasmid encoding HLA-B7 heavy chain and β2-microglobulin and is formulated with DMRIE-DOPE is Allovectin®.

The present invention further provides an immunotherapeutic composition containing (a) an antibody recognizing CTLA-4, PD-1, PD-L1, PD-L2, CD40, OX40, CD137, GITR, ILT2, or ILT3, in association with (b) one or more immunostimulatory therapeutic nucleic acid(s) having coding sequences for immunostimulatory proteins or peptides such as alloantigen(s), such as HLA-B7 (alone or in combination with class I major histocompatibility complex (MHC) antigens in addition to class II MHC and blood group antigens β2 microglobulins), and (c) a pharmaceutically acceptable carrier. In some embodiments, the antibody is an isolated fully-human monoclonal antibody. In some aspects, the immunotherapeutic composition contains an antibody recognizing CTLA-4, and one or more immunostimulatory therapeutic nucleic acid molecules(s) having coding sequences HLA-B7 and β2 microglobulin.

A binding component according to the present invention can be any binding component (e.g., an isolated fully-human monoclonal antibody) as set forth in U.S. Pat. No. 8,017,114 which is incorporated in its entirety herein. Alternatively, the binding components of the present invention may be blocking ligands, macromolecules (e.g., proteins or peptides, or nucleic acid molecules) or small molecules capable of binding to CTLA-4, PD-1, PD-L1, PD-L2, CD40, OX40, CD137, GITR, ILT2, or ILT3 and by way of this binding (e.g., through physical or steric effects) enhancing the activation of T cells or other immune cells.

An alloantigen according to the present invention may comprise class I major histocompatibility complex (MHC) antigens, as set forth in U.S. Pat. No. 5,910,488 which is incorporated in its entirety herein.

The invention also provides the immunostimulatory therapeutic nucleic acid molecules(s) optionally formulated with a pharmaceutical composition containing a transfer-facilitating vehicle. The vehicle may comprise a transfection-facilitating cationic lipid formulation. The cationic lipid formulation may be DMRIE-DOPE.

The invention further provides a method for treating a disorder, in an subject, characterized as being responsive to the stimulation of T-cell immunity, including the step of administering a vector into tissue or cells of the subject, wherein the vector comprises genetic material encoding one or more cistrons capable of expressing one or more proteins or peptides that stimulate T-cell immunity against the tissue or cells, such that the protein or proteins or peptide or peptides are expressed resulting in the treatment of the disorder followed by the administration of a binding agent.

The invention further provides a method for treating a disorder, in an subject, characterized as being responsive to the stimulation of T-cell immunity, including the administering a vector into tissue or cells of the subject, wherein the vector comprises genetic material encoding one or more cistrons capable of expressing one or more proteins or peptides that stimulate T-cell immunity against the tissue or cells, such that the protein or proteins or peptide or peptides are expressed to elicit an immune response and the administration of a binding agent, such as any humanized antibody recognizing CTLA-4, PD-1, PD-L1, PD-L2, CD40, OX40, CD137, GITR, ILT2, or ILT3.

In some embodiments, the disorder treated by a method of the present invention is cancer. In some embodiments, the cancer is selected from the group consisting of melanoma, squamous cell carcinoma, basal cell carcinoma, breast cancer, head and neck carcinoma, thyroid carcinoma, soft tissue sarcoma, bone sarcoma, testicular cancer, prostatic cancer, ovarian cancer, bladder cancer, skin cancer, brain cancer, angiosarcoma, hemangiosarcoma, mast cell tumor, primary hepatic cancer, lung cancer, pancreatic cancer, gastrointestinal cancer, renal cell carcinoma, hematopoietic neoplasia, and metastatic cancer thereof. In some embodiments, the cancer is melanoma, squamous cell carcinoma, or basal cell carcinoma. In particular embodiments, the cancer is melanoma.

An embodiment of the present invention includes a method for treating or preventing a medical condition in a subject (e.g., of melanoma, squamous cell carcinoma, breast cancer, head and neck carcinoma, thyroid carcinoma, soft tissue sarcoma, bone sarcoma, testicular cancer, prostatic cancer, ovarian cancer, bladder cancer, skin cancer, brain cancer, angiosarcoma, hemangiosarcoma, mast cell tumor, primary hepatic cancer, lung cancer, pancreatic cancer, gastrointestinal cancer, renal cell carcinoma, hematopoietic neoplasia, and metastatic cancer thereof) including administering a composition including: (a) a therapeutically effective amount of one or more binding components such as any antibody recognizing CTLA-4, PD-1, PD-L1, PD-L2, CD40, OX40, CD137, GITR, ILT2, or ILT3, preferably an isolated fully-human monoclonal antibody, in association with (b) a therapeutically effective amount of one or more vector(s) capable of expressing protein(s) or peptide(s) that stimulate T-cell immunity against tissues or cells and (c) a pharmaceutically acceptable carrier.

The present invention also provides a kit including (a) one or more binding components such as any antibody recognizing CTLA-4, PD-1, PD-L1, PD-L2, CD40, OX40, CD137, GITR, ILT2, or ILT3; in association with (b) one or more immunostimulatory therapeutic nucleic acid(s) capable of expressing protein(s) or peptide(s) that stimulate T-cell immunity against tissues or cells formulated in a pharmaceutically acceptable carrier. The protein(s) or peptides may comprise class I major histocompatibility complex (MHC) antigens, β2-microglobulins, or cytokines. The MHC antigen may be foreign to the subject. The MHC antigen may be HLA-B7. The binding component can be in a separate container from the vector.

In some embodiments, the kit contains a first container including a controlled release formulation of an antibody selected from the group consisting of ipilimumab, BMS-936558, BMS-936559, BMS-663513 or urelumab, CT-011 or pidilizumab, MK-3475, MPDL3280A or RG7446, CP-870,893, TRX518, or TRX385, in which the formulation contains an amount of antibody effective to treat or reduce and/or prevent melanoma, and a second container containing an immunostimulatory therapeutic nucleic acid molecule and a pharmaceutically acceptable carrier. In some embodiments of the kit, the immunostimulatory therapeutic nucleic acid molecule and pharmaceutically acceptable carrier are a controlled release formulation of a plasmid encoding HLA-B7 heavy chain and β2-microglobulin, formulated with DMRIE-DOPE in an amount effective to treat or reduce and/or prevent melanoma. The kits may further include a puncture needle or catheter. Any of the kits may also contain a package insert.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

### In the Drawings:

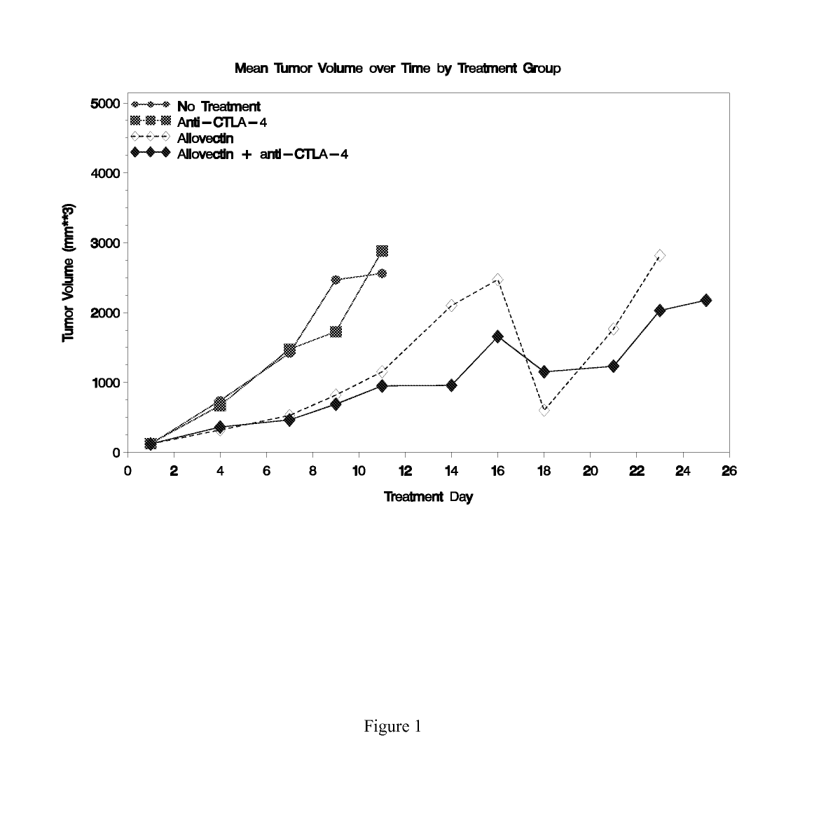

FIG. 1 presents mean tumor volumes over time for Groups 1-4, and illustrates the anti-tumor effect of the immunotherapeutic composition treatment.

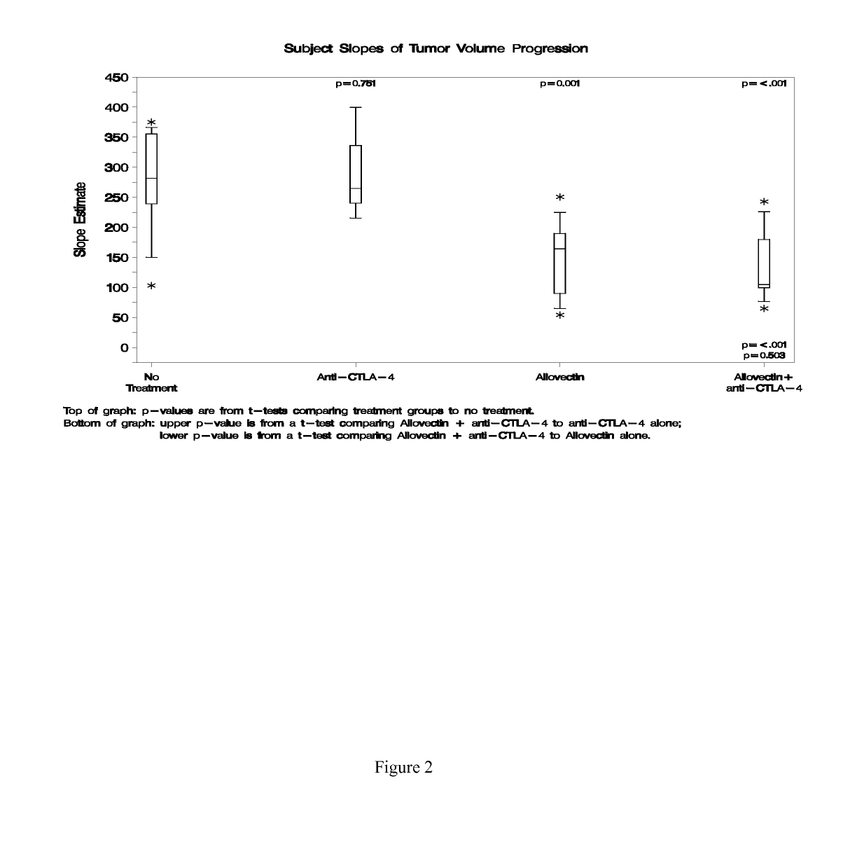

FIG. 2 represents the relationship of tumor volume between Groups 1-4.

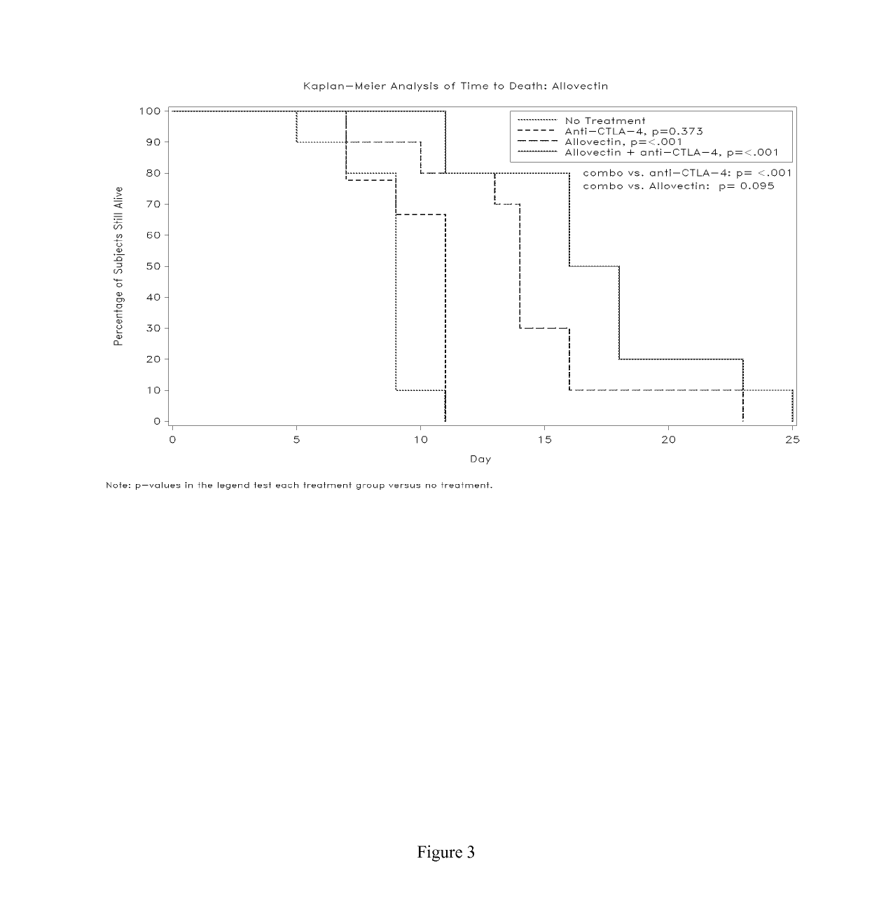

FIG. 3 graphically displays the survival curves for Groups 1-4.

### DETAILED DESCRIPTION OF THE INVENTION

The present invention provides synergistic combinations of immunotherapies and methods for treating disorders or medical conditions that are characterized by a down-regulation of MHC class I, such as cancer. The immunotherapeutic compositions of the invention, which can be used to treat the medical conditions, include one or more fully-human monoclonal antibodies recognizing CTLA-4, PD-1, PD-L1, PD-L2, CD40, OX40, CD137, GITR, ILT2, or ILT3, such as but not limited to ipilimumab in association with one or more immunostimulatory therapeutic nucleic acid(s), capable of expressing protein(s) or peptide(s) that stimulate T-cell immunity against tissues or cells formulated in a pharmaceutically acceptable carrier, such as but not limited to Allovectin®. The protein(s) or peptides may comprise class I major histocompatibility complex (MHC) antigens, β2-microglobulins, or cytokines. The MHC antigen may be foreign to the subject to which the immunotherapeutic composition is administered. The MHC antigen may be HLA-B7.

The “immunotherapeutic compositions” of the invention include the binding component and the immunostimulatory therapeutic nucleic acid component “in association” with one another. The term “in association” indicates that the components of the pharmaceutical compositions of the present invention can be formulated into a single composition for simultaneous delivery or formulated separately into two or more compositions (e.g., a kit). Furthermore, each component of the pharmaceutical composition of the invention can be administered to a subject at the same time in concomitant injections (separate) or at a different time than when the other component is administered (sequential injections (in any order)); for example, each administration may be given non-simultaneously at several intervals over a given period of time. Preferably, the immunostimulatory therapeutic nucleic acid component is administered first according to the preferred recommended dose and schedule, which is weekly for six weeks followed by a rest period of two to three weeks, followed by the administration of the binding component according to the recommended dose and schedule, which for example for ipilimumab is 3 mg/kg as an intravenous infusion every 3 weeks for a total of four doses. Moreover, the separate components may be administered to a subject by the same or by a different route (e.g., intratumoral, intravenous).

The immunotherapeutic compositions and methods of use of the invention provide a particularly effective means for treating diseases marked by reduced expression of MHC molecules. Surprisingly, the Examples described below demonstrate that the therapeutic efficacy of both the binding component and the immunostimulatory therapeutic nucleic acid component of the invention when administered in association demonstrate synergy.

“Synergy” and variations thereof refer to activity (e.g., immunostimulatory activity) of administering a combination of compounds that is greater than the additive activity of the compounds If administered individually.

As used herein, an “immunostimulatory therapeutic molecule” is any molecule (e.g., small molecule, protein, peptide, nucleic acid molecule, or antibody) that is administered to a patient to stimulate the patient's immune system for the purpose of treating a disease (e.g., a cancer or infectious disease). As used herein, an “immunostimulatory therapeutic nucleic acid” is a subset of an immunostimulatory therapeutic molecule and is any expression vector that when administered to a patient expresses protein(s) or peptide(s) that stimulate the patient's immune system for the purpose of treating a disease (e.g., a cancer or infectious disease). In particular, the invention relates to an immunostimulatory therapeutic nucleic acid or expression vector having the coding sequences of one or more alloantigen(s) with or without the coding sequence of one or more accessory molecules. In a specific embodiment, the expression vector is a bicistronic plasmid encoding human HLA-B7 heavy chain and chimpanzee β2-microglobulin as disclosed in U.S. Pat. No. 5,910,488, which is hereby incorporated herein in its entirety.

A coding sequence is “under the control of”, “functionally associated with” or “operably associated with” transcriptional and translational control sequences in a cell when the sequences direct RNA polymerase mediated transcription of the coding sequence into RNA, preferably mRNA, which then may be trans-RNA spliced (if it contains introns) and, optionally, translated into a protein encoded by the coding sequence.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The terms “vector”, “cloning vector” and “expression vector” mean the vehicle (e.g., a plasmid) by which a DNA or RNA sequence can be introduced into a host cell, so as to transform the host and, optionally, promote expression and/or replication of the introduced sequence. Vectors may contain nucleic acid molecules encoding one or more proteins or peptides. In preferred embodiments, the vector is a plasmid.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

### I. Binding Component

CTLA-4 (CD152) is expressed on T cells. When CD152 binds to CD80 or CD86 (e.g., as expressed on antigen presenting cells), a T cell inhibitory signal is generated. CD28, also expressed by T cells, likewise binds to CD80 and CD86, however this binding leads to the opposite effect, the generation of a T cell activation signal. Blocking CD152 activity, for example with neutralizing antibodies, therefore favors T cell activation in two ways. First, it reduces or eliminates the generation of a T cell inhibitory signal. Second, by freeing CD80 and CD86 to bind to CD28, it enhances the opportunity for delivery of T cell activation signals. In an analogous manner, PD-1 (CD279) expressed on activated T cells, B cells, and macrophages is capable of down-regulating T cell activation. The primary binding partners for PD-1 are PD-L1 (CD274) and PD-L2 (CD273). PD-L1 is constitutively expressed on many cell types, including tumor cells, whereas PD-L2 is inducible on dendritic cells, T cells and B cells. Engagement of PD-1 by PD-L1 or PD-L2 negatively regulates immune responses in a manner similar to but distinct from that produced following CTLA-4 binding to CD80 or CD86 (in part based on distinct expression patterns between these molecules). Like CTLA-4, PD-L1 is also capable of binding CD80, and therefore through competition for CD80 binding PD-L1 may also reduce CD28-mediated costimulatory signals. Other molecules capable of generating inhibitory signals in T cells and/or other immune cells (such as natural killer cells) include two members of the immunoglobulin-like transcript family, ILT2 and ILT3, whose ligands include MHC class I molecules. Blocking ILT2 and ILT3 binding should enhance T cell activation and/or survival in a manner analogous to blocking CTLA-4, PD-1, PD-L1, or PD-L2. Finally, rather than blocking immunoinhibitory molecules, engagement of immunostimulatory molecules (e.g., by agonist monoclonal antibodies) should have the same overall effect of enhancing immune cell activity and as a consequence anti-tumor responses. These latter molecules include CD40, OX40, CD137, and GITR.

The binding component of the immunotherapeutic composition of the present invention includes any composition which binds specifically to a molecule that regulates the activity of immune cells, such as, but not limited antibodies recognizing CTLA-4, PD-1, PD-L1, PD-L2, CD40, OX40, CD137, GITR, ILT2, or ILT3. Examples of these include the anti-CTLA-4 antibody ipilimumab (marketed by Bristol-Meyers Squibb as Yervoy®), the anti-PD-1 antibody BMS-936558 (under development by Bristol-Meyers Squibb, and also known as MDX-1106 or ONO-4538), the anti-PD-1 antibody CT-011 or pidilizumab (under development by CureTech), the anti-PD-1 antibody MK-3475 (under development by Merck, and also known as SCH 900475), the anti-PD-L1 antibody BMS-936559 (under development by Bristol-Meyers Squibb, and also known as MDX-1105), the anti-PD-L1 antibody MPDL3280A or RG7446 (under development by Genentech/Roche), the anti-CD137 monoclonal antibody BMS-663513 or urelumab (under development by Bristol-Meyers Squibb), the anti-CD40 agonist monoclonal antibody CP-870,893 (under development by Pfizer), the anti-GITR antibody TRX518 (formerly under development by Tolerx) and the anti-ILT3 antibody TRX385 (formerly under development by Tolerx).

A binding component or agent refers to a molecule that binds with specificity to an immunoregulatory molecule such as but not limited to CTLA-4, PD-1, PD-L1, PD-L2, CD40, OX40, CD137, GITR, ILT2, or ILT3, e.g., in a ligand-receptor type fashion or an antibody-antigen interaction e.g., proteins which specifically associate with immunoregulatory molecules such as but not limited to CTLA-4, PD-1, PD-L1, PD-L2, CD40, OX40, CD137, GITR, ILT2, or ILT3, e.g., in a natural physiologically relevant protein-protein interaction, either covalent or non-covalent. The term “binding component” includes small organic molecules, nucleic acids and polypeptides, such as a full antibody (preferably an isolated human monoclonal antibody) or antigen-binding fragment thereof of the present invention. Preferably the binding component of the present invention is ipilimumab a fully human anti-CTLA-4 monoclonal antibody (also known as 10D1 as disclosed in U.S. Pat. No. 8,017,144, which is hereby incorporated herein in its entirety) approved by the FDA for use in melanoma and marketed as Yervoy.

CTLA-4, PD-1, PD-L1, PD-L2, CD40, OX40, CD137, GITR, ILT2, or ILT3 activity could be blocked or enhanced in ways other than by the use of neutralizing antibodies. One could, for example, administer blocking ligands, macromolecules (e.g., proteins or peptides) or small molecules capable of binding to the molecule of interest and by way of this binding (e.g., through physical or steric effects) preventing their binding to other molecules. These blocking ligands, for example, could be based on CD80 or CD86 but lacking in their ability to trigger CD152 signaling. In this case, it would be preferable if these blocking ligands were not capable of binding CD28, so as to preserve functioning of the CD28-mediated T cell activation pathway.

One could also achieve the same overall effect as CTLA-4, PD-1, or PD-L1 blockade by enhancing CD28-mediated T cell activation. This could be accomplished, for example, by the administration of CD28 agonists (e.g., antibodies or macromolecules such as proteins or peptides or small molecules that trigger the appropriate cell signaling). Selection of the proper agonist would be important, as some CD28 agonists (e.g., so-called superagonists such as the antibody TGN1412) can trigger excessive and unwanted activation of multiple T cell and leukocyte populations, leading to the syndrome known as cytokine storm.

Immune activation can also be triggered through the interaction of CD40 and CD154 (also known as CD40 ligand or CD40L). CD40 is expressed by antigen presenting cells (e.g., macrophages) and CD154 by T cells, and their interaction leads to the activation of the CD40-expressing cell. Therefore, administration of an immunostimulatory therapeutic nucleic acid, such as but not limited to Allovectin® along with immunomodulators that lead to enhanced CD40-CD154 signaling would lead to increased immune activation and as a result increased anti-tumor activity. These immunomodulators could include CD40 ligands, for example macromolecules (e.g., proteins or peptides) or small molecules based on CD154 that are capable of binding to and triggering cell signaling by CD40 or agonist monoclonal antibodies capable of binding to and signaling through CD40. A similar approach could also be taken to enhance immune activation triggered through OX40 and its ligand OX40L, or CD137 and its ligand CD137L, or GITR and its ligand GITRL, through the administration of immunomodulators specific for these molecules (such as OX40L, or CD137L or GITRL, or macromolecules such as peptides or agonist monoclonal antibodies that are capable of binding to and signaling through OX40 or CD137 or GITR).

A. Effective Dosages of Binding Component

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the binding components are dictated by and directly dependent on (a) the unique characteristics of the binding component and the particular therapeutic effect to be achieved, and (b) the limitations inherent in the art of compounding such a binding component for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Regardless of the route of administration selected, the binding components, which may be used in a suitable hydrated form are formulated into pharmaceutically acceptable dosage forms by conventional methods known to those of skill in the art.

Actual dosage levels of the binding components of the present invention can be varied so as to obtain an amount of the binding component which is effective to achieve the desired therapeutic response for a particular patient, receiving the immunotherapeutic composition, and mode of administration, without being toxic to the patient. The selected dosage level depends upon a variety of pharmacokinetic factors including the activity of the particular binding components employed, or the ester, salt or amide thereof, the route of administration, the time of administration, the rate of excretion of the particular binding components being employed, the duration of the treatment, the immunostimulatory therapeutic nucleic acid used in combination with the particular binding components employed, the age, sex, weight, condition, general health and prior medical history of the patient being treated, and like factors.

A physician or veterinarian can start doses of the binding components employed in the pharmaceutical composition at levels lower than that required to achieve the desired therapeutic effect and gradually increase the dosage until the desired effect is achieved. In general, a suitable daily dose of a binding component is that amount of the binding component which is the lowest dose effective to produce a therapeutic effect. Such an effective dose generally depends upon the factors described above. It is preferred that administration be intravenous, intramuscular, intraperitoneal, intratumoral, or subcutaneous, or administered proximal to the site of the target. If desired, the effective daily dose of binding components can be administered as two, three, four, five, six or more sub-doses administered separately at appropriate intervals throughout the day, optionally, in unit dosage forms.

Effective doses of the binding components, for the treatment of immune-related conditions and diseases described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy.

For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. Preferably, the administration of the antibody is according to the recommended dose and schedule, which for example for ipilimumab is 3 mg/kg as an intravenous infusion every 3 weeks for a total of four doses. In some methods, two or more monoclonal antibodies with different binding specificities are administered simultaneously, in which case the dosage of each antibody administered falls within the ranges indicated. Antibody or antibodies are usually administered on multiple occasions. Intervals between single dosages can be weekly, monthly or yearly. Intervals can also be irregular as indicated by measuring blood levels of monoclonal antibodies in the patient. In some methods, dosage is adjusted to achieve a plasma antibody concentration of 1-1000 μg/ml and in some methods 25-300 μg/ml. Alternatively, antibody or antibodies can be administered as a sustained release formulation, in which case less frequent administration is required. Dosage and frequency vary depending on the half-life of the antibody or antibodies in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Some human sequence antibodies and human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, See, e.g., U.S. Pat. Nos. 4,522,811; 5,374,548; and 5,399,331. The liposomes may comprise one or more moieties which are selectively transported into specific cells or organs, thus enhance targeted drug delivery (See, e.g., V. V. Ranade, J. Clin. Pharmacol. 29:685 (1989)). Exemplary targeting moieties include folate or biotin (See, e.g., U.S. Pat. No. 5,416,016 to Low et al.); mannosides (Umezawa, et al., Biochem. Biophys. Res. Commun. 153:1038 (1988)); antibodies (P. G. Bloeman, et al. FEBS Lett. 357:140 (1995); M. Owais et al. Antimicrob. Agents Chemother. 39:180 (1995)); surfactant protein A receptor (Briscoe, et al. Am. J. Physiol. 1233:134 (1995)), different species of which may comprise the formulations of the inventions, as well as components of the invented molecules; p 120 (Schreier et al., J. Biol. Chem. 269:9090 (1994)); See also K. Keinanen; M. L. Laukkanen, FEBS Lett. 346:123 (1994); J. J. Killion, et al., Immunomethods 4:273 (1994). In some methods, the binding components of the immunotherapeutic invention are formulated in liposomes; in a more preferred embodiment, the liposomes include a targeting moiety. In some methods, the binding component in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

For therapeutic applications, the binding components are administered to a patient suffering from established disease in an amount sufficient to arrest or inhibit further development or reverse or eliminate, the disease, its symptoms or biochemical markers. For prophylactic applications, the pharmaceutical compositions are administered to a patient susceptible or at risk of a disease in an amount sufficient to delay, inhibit or prevent development of the disease, its symptoms and biochemical markers. An amount adequate to accomplish this is defined as a “therapeutically-” or “prophylactically-effective dose.” Dosage depends on the disease being treated, the subject's size, the severity of the subject's symptoms, and the particular composition or route of administration selected. Specifically, in treatment of tumors, a “therapeutically effective dosage” can inhibit tumor growth by at least about 20%, or at least about 40%, or at least about 60%, or at least about 80% relative to untreated subjects. The ability of a compound to inhibit cancer can be evaluated in an animal model system predictive of efficacy in human tumors. Alternatively, this property of a binding component can be evaluated by examining the ability of the binding component to inhibit by conventional assays in vitro. A therapeutically effective amount of a binding component can decrease tumor size, or otherwise ameliorate symptoms in a subject. Ideally, reduced levels of monoclonals can be used with Allovectin, thereby reducing the risk of monoclonal-induced toxicity but still offering synergistic anti-tumor responses.

The binding component should be sterile and fluid to the extent that the binding component is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable binding components can be brought about by including in the binding component an agent which delays absorption, for example, aluminum monostearate or gelatin.

B. Routes of Administration of Binding Component

Pharmaceutically acceptable carriers include solvents, dispersion media, coatings, antibacterial and antifungal agents, isotonic and absorption delaying agents, and the like that are physiologically compatible. The carrier can be suitable for intravenous, intramuscular, subcutaneous, parenteral, spinal or epidermal administration (e.g., by injection or infusion). Depending on the route of administration, the binding component, i.e., antibody, bispecific and multispecific molecule, may be coated in a material to protect the binding component from the action of acids and other natural conditions that may inactivate the compound.

A “pharmaceutically acceptable salt” refers to a salt that retains the desired biological activity of the binding component and does not impart any undesired toxicological effects (See, e.g., Berge, S. M., et al., J. Pharm. Sci. 66:1-19 (1977)). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′ dibenzylethylenediamine, N-methylglucamine, chloroprocaine, choline, diethanolamine, ethylenediamine, procaine and the like.

A binding component of the present invention can be administered by a variety of methods known in the art. The route and/or mode of administration vary depending upon the desired results. The binding component can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, 1978. Pharmaceutical compositions are preferably manufactured under GMP conditions.

To administer a binding component of the invention by certain routes of administration, it may be necessary to coat the binding component with, or co-administer the binding component with, a material to prevent its inactivation. For example, the binding component may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan, et al., J. Neuroimmunol. 7:27 (1984)).

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the binding component, use thereof with the binding components of the invention is contemplated.

Sterile injectable solutions can be prepared by incorporating the binding component in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Binding components can also be administered with medical devices known in the art. For example, in a preferred embodiment, a binding component of the immunotherapeutic composition of the invention can be administered with a needleless hypodermic injection device, such as the devices disclosed in, e.g., U.S. Pat. No. 5,399,163, 5,383,851, 5,312,335, 5,064,413, 4,941,880, 4,790,824, or 4,596,556. Examples of implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

C. Formulation of Binding Component

For the binding components, formulations include those suitable for oral, nasal, topical (including buccal and sublingual), rectal, vaginal and/or parenteral administration. The formulations can conveniently be presented in unit dosage form and may be prepared by any methods known in the art of pharmacy. The amount of binding component which can be combined with a carrier material to produce a single dosage form vary depending upon the subject being treated, and the particular mode of administration. The amount of binding component which can be combined with a carrier material to produce a single dosage form generally be that amount of the binding component which produces a therapeutic effect. Generally, out of one hundred percent, this amount range from about 0.01 percent to about ninety-nine percent of active ingredient, from about 0.1 percent to about 70 percent, or from about 1 percent to about 30 percent.

The phrases “parenteral administration” and “administered parenterally” mean modes of administration other than enteral and topical administration, usually by injection, and includes, without limitation, intravenous, intratumoral, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the binding components include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

### II. Immunostimulatory Therapeutic Nucleic Acid Component and Delivery

As discussed previously, most malignancies arise in immunocompetent hosts, suggesting that reduced expression of MHC molecules may provide a mechanism to escape detection by the immune system. Consequently, the immunostimulatory therapeutic nucleic acid component is capable of expressing alloantigen(s) that stimulate T-cell immunity against tissues or cells. The expressed alloantigen may comprise class I or class II major histocompatibility complex (MHC) antigens. The MHC antigen may be foreign to the subject. The MHC antigen may be HLA-B7. The alloantigen may also compromise blood group antigens. Alternatively, the immunostimulatory nucleic acid component could be capable of expressing protein(s) or peptide(s) that could serve to restore or stimulate or enhance immune functioning, such as β2 microglobulins or cytokines. For example, cytokines such as IFN-γ and TNF are capable of increasing MHC expression, as well as stimulating immune cell activity.

When the alloantigen is expressed in the mammal, the expression produces a result selected from alleviation of the cancer, reduction of size of a tumor associated with the cancer, elimination of a tumor associated with the cancer, prevention of metastatic cancer, prevention of the cancer and stimulation of effector cell immunity against the cancer.

Preferably, the immunostimulatory therapeutic nucleic acid component of the present invention is composed of a bicistronic plasmid (preferably encoding HLA-B7 heavy chain and β2-microglobulin) formulated with a cationic lipid-based system (DMRIE-DOPE), also known as Allovectin®. Without wishing to be bound by any particular theory, Allovectin® is believed to act through multiple mechanisms of action (MOA): (i) induction of anti-tumor T cells following tumor cell expression of the alloantigen HLA-B7 in HLA-B7 negative patients, (ii) induction of anti-tumor T cells following restoration of tumor MHC class I expression and antigen presentation, and (iii) recruitment of immune cells into tumors through the pro-inflammatory action of DNA-lipid complexes. Generation of anti-tumor T cells drives the destruction of not only those tumor sites directly injected with Allovectin®, but also distal lesions and metastases.

A. Cationic Liposomes and Vehicles for Immunostimulatory Therapeutic Nucleic Acid Component Delivery

The transfer of the optimized immunostimulatory therapeutic nucleic acid component provided herein into cells or tissues of subjects may be accomplished by injecting naked DNA or facilitated by using vehicles, such as, for example, viral vectors, ligand-DNA conjugates, adenovirus-ligand-DNA conjugates, calcium phosphate, and liposomes. Transfer procedures are art-known, such as, for example, transfection methods using liposomes and infection protocols using viral vectors, including retrovirus vectors, adenovirus vectors, adeno-associated virus vectors, herpes virus vectors, vaccinia virus vectors, polio virus vectors, and sindbis and other RNA virus vectors.

According to one embodiment of the invention, the immunostimulatory therapeutic nucleic acid component provided herein are complexed with cationic liposomes or lipid vesicles. Cationic or positively charged liposomes are formulations of cationic lipids (CLs) in combination with other lipids. The formulations may be prepared from a mixture of positively charged lipids, negatively charged lipids, neutral lipids and cholesterol or a similar sterol. The positively charged lipid can be one of the cationic lipids, such as DMRIE, described in U.S. Pat. No. 5,264,618, which is hereby incorporated by reference, or one of the cationic lipids DOTMA, DOTAP, or analogues thereof, or a combination of these. Alternatively, the cationic lipid may be GAP-DMORIE in combination with a co-lipid as described in U.S. Pat. No. 6,586,409. DMRIE is 1,2-dimyristyloxypropyl-3-dimethylhydroxyethyl ammonium bromide (see, e.g., J. Feigner, et al., J. Biol. Chem., 269, 1 (1994)) and is preferred.

Neutral and negatively charged lipids can be any of the natural or synthetic phospholipids or mono-, di-, or triacylglycerols. The natural phospholipids may be derived from animal and plant sources, such as phosphatidylcholine, phosphatidylethanolamine, sphingomyelin, phosphatidylserine, or phosphatidylinositol. Synthetic phospholipids may be those having identical fatty acid groups, including, but not limited to, dimyristoylphosphatidylcholine, dioleoylphosphatidylcholine, dipalmitoylphosphatidylcholine, distearoylphosphatidylcholine and the corresponding synthetic phosphatidylethanolamines and phosphatidylglycerols. The neutral lipid can be phosphatidylcholine, cardiolipin, phosphatidylethanolamine, mono-, di- or triacylglycerols, or analogues thereof, such as dioleoylphosphatidylethanolamine (DOPE), which is preferred. The negatively charged lipid can be phosphatidylglycerol, phosphatidic acid or a similar phospholipid analog. Other additives such as cholesterol, glycolipids, fatty acids, sphingolipids, prostaglandins, gangliosides, neobee, niosomes, oranyothernatural or synthetic amphophiles can also be used in liposome formulations, as is conventionally known for the preparation of liposomes.

Substitution of the cationic lipid component of liposomes can alter transfection efficiencies. Specifically, modification of the cationic species appears to be an important determinant in this process. A new formulation of cationic lipids is preferred in which a different cationic lipid, 1,2-dimyristyloxypropyyl-3-dimethylhydroxyetheyl ammonium bromide (DMRIE), is utilized with dioleoyl phosphatidylethanolamine (DOPE). This formulation has two properties which make it more suitable for transfections. First, it shows up to about a 7-fold increase in improved transfection efficiency compared to the formulation DC-cholesterol/DOPE in vitro.

Importantly, this DMRIE/DOPE formulation does not aggregate at high concentrations, in contrast to the DC-Chol liposome. This characteristic thus allows higher absolute concentrations of DNA and liposomes to be introduced into experimental animals without toxicity. Because of these properties, it now becomes possible to introduce 100-1000 times more DNA which could markedly improve gene expression in vivo.

A preferred molar ratio of DMRIE to DOPE is from about 9/1 to 1/9; a molar ratio of about 5:5 is particularly preferred.

Using conventional cationic lipid technology and methods, the lipid compositions can be used to facilitate the intracellular delivery of genetic material coding for therapeutically or immunogenically active proteins or peptides. Briefly, such methods include the steps of preparing lipid vesicles composed of cationic lipids and using these lipid vesicles to mediate the transfection or transport of therapeutically or immunogenically active agents into the cells. The intracellular transport may be accomplished by incorporating or encapsulating the agent in the lipid vesicle and contacting the cell with the lipid vesicles, as in conventional liposome methodology; or alternatively, by contacting the cells simultaneously with empty lipid vesicles, combining the cationic lipid formulations together with the agent, according to conventional transfection methodology. In the process of either strategy, the agent is taken up by the cell. The contacting step may occur in vitro or in vivo.

Such methods may be applied in the treatment of a disorder in an subject, including the step of administering a preparation having a cationic lipid formulation together with a pharmaceutically effective amount of immunostimulatory therapeutic nucleic acid component specific for the treatment of the disorder in the subject and permitting the agent to be incorporated into a cell, whereby the disorder is effectively treated. The immunostimulatory therapeutic nucleic acid component may be delivered to the cells of the subject in vitro or in vivo. The in vitro delivery of the immunostimulatory therapeutic nucleic acid component is carried out on cells that have been removed from an organism. The cells are returned to the body of the subject whereby the subject is treated. In contrast, in vivo delivery involves direct transduction of cells within the body of the subject to effect treatment. Cationic lipid mediated delivery of vectors encoding therapeutic agents can thus provide therapy for genetic disease by supplying deficient or missing gene products to treat any disease in which the defective gene or its product has been identified, such as Duchenne's dystrophy (Kunkel, L. and Hoffman, E. Brit. Med. Bull. 45(3):630-643 (1989)) and cystic fibrosis (Goodfellow, P. Nature, 341(6238):102-3 (1989)).

The cationic lipid mediated intracellular delivery described can also provide immunizing peptides. The above transfection procedures may be applied by direct injection of cationic lipid formulations together with a vector coding for an immunogen into cells of an animal in vivo or transfection of cells of an animal in vitro with subsequent reintroduction of the transduced cells into the animal. The ability to transfect cells with cationic lipids thus provides an alternate method for immunization. The gene for an antigen is introduced, by means of cationic lipid-mediated delivery, into cells of an animal. The transfected cells, expressing the antigen, are reinjected into the animal or already reside within the animal, where the immune system can respond to the antigen. The process can be enhanced by co-administration of either an adjuvant or cytokines such as lymphokines, or a gene coding for such adjuvants or cytokines or lymphokines, to further stimulate the lymphoid cells and other cells mediating the immune response.

Administration to patients diagnosed with neoplastic disease of DNA liposome complexes for the treatment of neoplasia involves, preferably, intratumoral injection, by needle and syringe or by catheter (see infra), of the complexes. Plasmid DNA in an amount ranging from about 0.1 microgram to about 5 g is administered in from about 0.15 nanoMolar to about 1.5 milliMolar liposome solution. In a preferred protocol, 0.1 ml of plasmid DNA (0.05-50 mg/ml) in lactated Ringer's solution is added to 0.1 ml of DMRIE/DOPE liposome solution (0.15-15 microMolar), and 0.8 ml of lactated Ringer's solution is added to the liposome DNA solution. In this preferred protocol, three aliquots of 0.2 ml each are injected into a nodule or one aliquot of 0.6 ml is applied by catheter. The patient, in this preferred protocol, is thus administered a dose ranging from about 3 microgram to about 3 milligram of DNA and from about 4.5 nanoMolar to about 4.5 microMolar DMRIE/DOPE. Doses are repeated at two-week intervals.

A combination, or any component thereof, of the invention can be incorporated into an immunotherapeutic composition, along with a pharmaceutically acceptable carrier, suitable for administration to a subject in vivo. The scope of the present invention includes immunotherapeutic compositions which may be administered to a subject by any route, such as a parenteral route (e.g., intratumoral injection, intravenous injection, intraarterial injection, subcutaneous injection or intramuscular injection). In one embodiment, the immunotherapeutic compositions of the invention comprises an antibody recognizing CTLA-4, PD-1, PD-L1, PD-L2, CD40, OX40, CD137, GITR, ILT2, or ILT3, such a but not limited to ipilimumab in association with an immunostimulatory therapeutic nucleic acid component that expresses one or more alloantigens, such as but not limited to, Allovectin®.

As stated above, the immunotherapeutic composition of the present invention comprises a synergistic combinations of components that include a binding component and an immunostimulatory therapeutic nucleic acid component “in association” with one another. The term “in association” indicates that the components of the immunotherapeutic compositions of the invention can be formulated into a single composition for simultaneous delivery or formulated separately into two or more compositions (e.g., a kit). For example, the scope of the present invention includes compositions including an antibody recognizing CTLA-4, PD-1, PD-L1, PD-L2, CD40, OX40, CD137, GITR, ILT2, or ILT3 formulated for parenteral administration (e.g., intravenous) to a subject and an immunostimulatory therapeutic nucleic acid component formulated for parenteral administration (e.g., intratumoral). Alternatively, both components of the immunotherapeutic composition can be formulated, separately or together, for parenteral delivery.

For general information concerning formulations, see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, New York; Lieberman, et al., (eds.) (1990) Pharmaceutical Dosage Forms: Tablets Dekker, New York; and Lieberman, et al., (eds.) (1990), Pharmaceutical Dosage Forms: Disperse Systems Dekker, New York, Kenneth A. Walters (ed.) (2002) Dermatological and Transdermal Formulations (Drugs and the Pharmaceutical Sciences), Vol. 119, Marcel Dekker.

Sterile injectable solutions can be prepared by incorporating an immunotherapeutic composition of the invention or any component thereof (e.g., binding component and/or immunostimulatory therapeutic nucleic acid component), in the required amount, in an appropriate solvent, optionally with one or a combination of ingredients enumerated above, as required, followed by sterilization microfiltration. Generally, dispersions are prepared by incorporating the active ingredient (e.g., binding component and/or immunostimulatory therapeutic nucleic acid component) into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional, desired ingredient from a previously sterile-filtered solution thereof.

The immunotherapeutic composition of the invention or any component thereof (e.g., binding component and/or immunostimulatory therapeutic nucleic acid component) may also be administered by inhalation. A suitable immunotherapeutic composition for inhalation may be an aerosol. An exemplary immunotherapeutic composition for inhalation of the invention or any component thereof may include: an aerosol container with a capacity of 15-20 ml containing the active ingredient (e.g., binding component and/or chemotherapeutic agent), a lubricating agent, such as polysorbate 85 or oleic acid, dispersed in a propellant, such as freon, preferably in a combination of 1,2-dichlorotetrafluoroethane and difluorochloromethane. Preferably, the composition is in an appropriate aerosol container adapted for either intranasal or oral inhalation administration.

### Dosage of the Immunotherapeutic Composition

Preferably, the immunotherapeutic composition of the invention is administered to a subject at a “therapeutically effective dosage” or “therapeutically effective amount” which preferably inhibits a disease or condition (e.g., tumor growth) to any extent-preferably by at least about 20%, more preferably by at least about 40%, even more preferably by at least about 60%, and still more preferably by at least about 80%-100% relative to untreated subjects. The ability of the immunotherapeutic composition of the present invention or any component thereof to inhibit cancer can be evaluated in an animal model system predictive of efficacy in human tumors. Alternatively, this property can be evaluated by examining the ability of the immunotherapeutic composition of the invention or any component thereof to inhibit tumor cell growth in vitro by assays well-known to the skilled practitioner. One of ordinary skill in the art would be able to determine such amounts based on such factors as the subject's size, the severity of the subject's symptoms, and the particular composition or route of administration selected.

Dosage regimens may be adjusted to provide the optimum desired response (e.g., a therapeutic response). For example, a dose may be administered, several divided doses may be administered over time or the dose may be proportionally reduced or increased as indicated by exigencies of the therapeutic situation. It is especially advantageous to formulate parenteral compositions in dosage unit form for ease of administration and uniformity of dosage. A physician or veterinarian having ordinary skill in the art can readily determine and prescribe the effective amount of the pharmaceutical composition required. For example, the physician or veterinarian could preferably start by administering the immunostimulatory therapeutic nucleic acid component first according to the preferred recommended dose and schedule which is weekly for six weeks followed by a rest period of two to three weeks followed by the administration of the antibody recognizing CTLA-4, PD-1, PD-L1, PD-L2, CD40, OX40, CD137, GITR, ILT2, or ILT3 according to the recommended dose and schedule, which for example in the case of ipilimumab is 3 mg/kg as an intravenous infusion every 3 weeks for a total of four doses. Moreover, the separate components may be administered to a subject by the same or by a different route (e.g., intratumoral, orally, intravenously, intratumorally). If a patient is already receiving the binding component according to the prescribed regiment then the immunostimulatory therapeutic nucleic acid component would be added to the regiment.

In an alternative embodiment IL-2 is given after the immunostimulatory therapeutic nucleic acid component and before antibodies recognizing CTLA-4, PD-1, PD-L1, PD-L2, CD40, OX40, CD137, GITR, ILT2, or ILT3 in order to optimize the opportunity for T cell activation and/or proliferation. Alternatively, IL-2 and/or the immunostimulatory therapeutic nucleic acid component and/or antibodies recognizing CTLA-4, PD-1, PD-L1, PD-L2, CD40, OX40, CD137, GITR, ILT2, or ILT3 could be delivered concurrently. This regiment would be beneficial when steroids are used.

In yet another embodiment subjects will receive intralesional injection(s) of Allovectin® once a week for six consecutive weeks into a single lesion or into multiple lesions followed by three weeks of observation and evaluation. Subjects with stable or responding disease will receive additional cycles starting on Weeks 9, 17, 25, etc., until disease progression, complete response or unacceptable toxicity. The maximum number of cycles before surgery for the subjects with stable or responding disease will be six at the discretion of the investigator.

After Allovectin® neoadjuvant treatment patients will undergo complete surgical resection, followed with adjuvant interferon treatment. Patients will receive standard outpatient induction therapy (IFN-α-2b 20 million units/m² per day intravenously [IV] 5 days per week) for 4 weeks, followed by standard outpatient maintenance therapy (10 million units/m², subcutaneously [SC], 3 times per week), administered for 48 weeks.

Primary efficacy will be assessed by lesion response and time to disease recurrence. Lesions will be measured by any of the following methods: CT, MRI, or physical exam. Investigators will be instructed to use the same method of measurement on subsequent measurements when possible. All responders will be confirmed with a complete disease staging and measurements at least four weeks following the first evidence of response. Lesion response will be assessed by Modified RECIST Criteria (Response Evaluation Criteria in Solid Tumors).

Safety assessments will include vital signs, clinical laboratory tests, physical examinations, adverse events monitoring, and review of concomitant medication usage.

The effectiveness of the immunotherapeutic composition of the present invention can be determined, for example, by determining whether a tumor being treated in the subject shrinks or ceases to grow. The size of tumor can be easily determined, for example, by X-ray, magnetic resonance imaging (MRI) or visually in a surgical procedure.

In general, a suitable daily dose of the immunotherapeutic composition of the invention thereof may be that amount which is the lowest dose effective to produce a therapeutic effect. Such an effective dose will generally depend upon the factors described above. It is preferred that administration be by injection, preferably proximal to the site of the target (e.g., tumor). If desired, a therapeutically effective daily dose of the immunotherapeutic composition of the present invention hereof may be administered as two, three, four, five, six or more sub-doses administered separately at appropriate intervals throughout the day. In an embodiment, a “therapeutically effective dosage” of a chemotherapeutic agent is as set forth in the Physicians' Desk Reference 2003 (Thomson Healthcare; 57^{th} edition (Nov. 1, 2002)) which is herein incorporated by reference.

The present invention also provides kits including the components of the compositions of the invention in kit form. A kit of the present invention includes one or more components including, but not limited to, a binding component, as discussed herein, which specifically binds CTLA-4, PD-1, PD-L1, PD-L2, CD40, OX40, CD137, GITR, ILT2, or ILT3 in association with one or more additional components including, but not limited to, an immunostimulatory therapeutic nucleic acid component, as discussed herein. The binding component and/or the immunostimulatory therapeutic nucleic acid component can be formulated as a pure composition or in combination with a pharmaceutically acceptable carrier, in an immunotherapeutic composition.

In one embodiment, a kit includes a binding component of the invention (e.g., an anti-CTLA-4 antibody, such as ipilimumab, or an antibody recognizing PD-1, PD-L1, PD-L2, CD40, OX40, CD137, GITR, ILT2, or ILT3) and an immunostimulatory therapeutic nucleic acid component thereof in another container (e.g., in a sterile glass or plastic vial).

In another embodiment of the invention, the kit comprises a composition of the invention, including a binding component (e.g., anti-CTLA-4 antibody, such as ipilimumab, or an antibody recognizing PD-1, PD-L1, PD-L2, CD40, OX40, CD137, GITR, ILT2, or ILT3) along with an immunostimulatory therapeutic nucleic acid component such as Allovectin® formulated together, optionally, along with a pharmaceutically acceptable carrier, in an immunotherapeutic composition, in a single, common container.

If the kit includes an immunotherapeutic composition for parenteral administration to a subject, the kit can include a device for performing such administration. For example, the kit can include one or more hypodermic needles or other injection devices as discussed above.

The kit can include a package insert including information concerning the immunotherapeutic compositions or individual component and dosage forms in the kit. Generally, such information aids patients and physicians in using the enclosed immunotherapeutic compositions and dosage forms effectively and safely. For example, the following information regarding the immunotherapeutic composition of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

All references cited herein, including patents, patent applications, and publications, are hereby incorporated by reference in their entireties, whether previously specifically incorporated or not.

The Examples provided herein suggest that a combined treatment approach using both Allovectin® and an antibody recognizing CTLA-4, PD-1, PD-L1, PD-L2, CD40, OX40, CD137, GITR, ILT2, or ILT3 would result in additive or greater efficacy given the distinct yet related mechanisms of action (MOAs) of these two immunotherapies. As a combination therapy, Allovectin® would first act to generate a tumor-reactive T cell repertoire. Antibodies recognizing CTLA-4, PD-1, PD-L1, PD-L2, CD40, OX40, CD137, GITR, ILT2, or ILT3 would then serve to maximally activate these cell populations. A murine melanoma model was used in the disclosed studies.

The invention is further illustrated by the following non-limiting examples. All scientific and technical terms have the meanings as understood by one with ordinary skill in the art. The specific examples which follow illustrate the methods in which the compositions of the present invention may be prepared and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### Example 1

VCL-1005, DMRIE/DOPE, and Allovectin® were prepared by Vical Incorporated. The bicistronic plasmid VCL-1005 (encoding human HLA-B7 heavy chain and chimpanzeeβ2-microglobulin) was formulated at 2 mg/mL in IVF-1 vehicle (0.9% saline containing 10 μL/mL glycerin), the lipids DMRIE and DOPE was mixed at a 1:1 molar ratio and adjusted to a total lipid concentration of 0.86 mg/mL in IVF-1, and Allovectin® was prepared as 2 mg/mL VCL-1005 formulated with 0.86 mg/mL DMRIE/DOPE in IVF-1. Hamster anti-murine-CTLA-4 (clone 9H10) and an isotype-matched control hamster IgG (clone SHG-1) were purchased as 1 mg/mL, azide-free solutions (BioLegend, San Diego, Calif.).

Animal studies were conducted by Piedmont Research Center (Morrisville, N.C.) according to guidelines recommended in the Guide for Care and Use of Laboratory Animals (National Academy Press, Washington, D.C.) under the oversight of an Institutional Animal Care and Use Committee. B16-F10 murine melanoma cells were maintained as exponentially growing cultures in RPMI-1640 medium containing 10% fetal bovine serum, and for implantation were harvested during log phase growth and resuspended in phosphate buffered saline (PBS). Female C57BL/6 mice (Charles River Laboratories, Wilmington, Mass.), 7 to 8 weeks old, were implanted subcutaneously on the right flank with 5×10⁶ B16-F10 cells in a 0.2 mL volume. Six days later, mice were randomized into groups (N=10, mean group tumor volume=119-120 mm³) and treatments were initiated (Day 1).

Treatment groups were: no treatment (control), Allovectin® (100 μg) plus SHG-1 or 9H10, VCL-1005 (100 μg) plus SHG-1 or 9H10, or DMRIE/DOPE (43 μg) plus SHG-1 or 9H10. Allovectin®, VCL-1005 and DMRIE/DOPE were delivered intratumorally (i.t.) as 50 μL volumes daily on Days 1-4 (qd×4). Antibodies (SHG-1 and 9H10) were delivered intraperitoneally (i.p.) as 100 μg on Day 1 and thereafter every 3 days (q3d) as 50 μg. Tumor dimensions were measured with calipers every three days, and tumor volume (TV, in mm³) calculated according to the formula: TV=(W²×L)/2, where W=tumor width and L=tumor length (in mm). Animals were monitored daily for survival and general clinical signs.

In order to determine if the magnitude tumor growth observed in the Allovectin® plus anti-CTLA-4 group was less than the sum of the corresponding effects of either treatment alone, tumor volume slope was used. This endpoint can be computed for each animal using the available tumor measurements, and does not require that the number and spacing of measurements be identical for all mice.

The groups to be used in determining a synergism effect were defined as Group 1 (no treatment), Group 2 (anti-CTLA-4 alone), Group 3 (Allovectin® alone), and Group 4 (Allovectin® plus anti-CTLA-4). Letting μl, μ2, μ3, and μ4 denote the mean slopes from groups 1-4, respectively, the parameter of interest for assessing synergism was: S=(μ4−μ1)−((μ2−μ1)+(μ3−μ1)). Using the slope for each mouse as the dependent variable, a one-way analysis of variance (ANOVA) model with treatment group as the factor was fit to the data. The parameter S was then estimated using this model.

The estimated value of S was calculated to be −29.6, indicating that the difference between the combination treatment and no treatment is less than the sum of the differences between each individual treatment and no treatment. The conclusion is that there was a synergistic effect observed for the combination of Allovectin® and anti-CTLA-4 in this mouse study. FIG. 1 presents mean tumor volumes over time for Groups 1-4, and illustrates the anti-tumor effect of the combination treatment.

Using the same ANOVA model, the slopes of Groups 1-4 were also compared. Two hypotheses were tested. For single treatment groups vs. combination groups in mean of slope, the test was μ4=μ2 and μ4=μ3. For each group vs. the no treatment group in mean of slope, the test was μ2=μ1 and μ3=μ1 and μ4=μ1.

It was found that tumor volume was significantly reduced in Group 4 (Allovectin® plus anti-CTLA-4) compared to Group 1 (no treatment, p<0.001) and Group 2 (anti-CTLA-4 alone, p<0.001). The tumor volume was also significantly reduced in Group 3 (Allovectin® alone) compared to Group 1 (p=0.001). These relationships are presented as FIG. 2.

Survival was also compared between groups. All mice died prior to last time point (Day 28), therefore there were no censored observations. FIG. 3 graphically displays the survival curves for Groups 1-4. The log-rank test was employed to test for statistical significance between groups, and showed that survival was significantly improved in Group 4 compared to both Group 1 and Group 2 (p<0.001). Survival was also statistically significantly improved in Group 3 compared to Group 1 (p<0.001).

The foregoing description is considered as illustrative only of the principles of the invention. Further, since numerous modifications and changes will readily occur to those skilled in the art, it is not desired to limit the invention to the exact construction and process as described above. Accordingly, all suitable modifications and equivalents may be resorted to falling within the scope of the invention as defined by the claims that follow.

The term “comprising”, which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

### Example 2

Anti-tumor activity may be confirmed using a study with the following or similar design. Solid B16-F10 tumors are established on the flank of C57/BL6 or B6D2F1 mice, and when tumors are palpable and approximately 100 mm³ in volume, animals are randomized to treatment groups. Treatment groups include: anti-PD-1, anti-PD-L1, Allovectin plus normal IgG (or an irrelevant antibody), Allovectin plus anti-PD-1, Allovectin plus anti-PD-L1, and non-treated tumor-bearing mice as controls. Allovectin is delivered by intratumoral injection as a 100 μg dose for four consecutive days (100 μg qd×4), and antibodies are delivered by intraperitoneal injection as 200 μg doses every 3 days until study end (200 μg q3 d). Antibodies are reactive with mouse PD-1 or PD-L1, such as the rat monoclonal antibodies RPM1-14 and 10F.9G2, respectively. Animals are followed for tumor volume (measured every 3 days using calipers) and survival; mean tumor volume slopes are compared between groups using a one-way ANOVA analysis, and survivals are compared by the log-rank test.

We claim:

1. An immunotherapeutic composition comprising (a) one or more binding component(s), in association with (b) one or more immunostimulatory therapeutic nucleic acid molecule(s) and, optionally, a pharmaceutically acceptable carrier.

2. The immunotherapeutic composition of claim 1, wherein said one or more binding component(s) is a molecule that binds with specificity to CTLA-4, PD-1, PD-L1, PD-L2, CD40, OX40, CD137, GITR, ILT2, or ILT3, or a molecule that binds with specificity to a ligand of CTLA-4, PD-1, PD-L1, PD-L2, CD40, OX40, CD137, GITR, ILT2, or ILT3.

3. The immunotherapeutic composition of claim 2, wherein said molecule that binds with specificity to CTLA-4, PD-1, PD-L1, PD-L2, CD40, OX40, CD137, GITR, ILT2, or ILT3 orsaid molecule that binds with specificity to a ligand of CTLA-4, PD-1, PD-L1, PD-L2, CD40, OX40, CD137, GITR, ILT2, or ILT3 is selected from the group consisting of a small organic molecule, a nucleic acid molecule, and a polypeptide.

4. The immunotherapeutic composition of claim 3, wherein said polypeptide is an antibody having specificity to CTLA-4, PD-1, PD-L1, PD-L2, CD40, OX40, CD137, GITR, ILT2, or ILT3; or an antibody having specificity to a ligand of CTLA-4, PD-1, PD-L1, PD-L2, CD40, OX40, CD137, GITR, ILT2, or ILT3; or an antigen-binding fragment thereof.

5. The immunotherapeutic composition of claim 4, wherein said antibody having specificity to CTLA-4, PD-1, PD-L1, PD-L2, CD40, OX40, CD137, GITR, ILT2, or ILT3, or to a ligand of CTLA-4, PD-1, PD-L1, PD-L2, CD40, OX40, CD137, GITR, ILT2, or ILT3, is a human monoclonal antibody.

6. The immunotherapeutic composition of claim 4, wherein the antibody has specificity to CTLA-4, PD-1, or PD-L1.

7. The immunotherapeutic composition of claim 6, wherein the antibody has specificity to CTLA-4.

8. The immunotherapeutic composition of claim 5, wherein said human monoclonal antibody is ipilimumab, BMS-936558, BMS-936559, BMS-663513, CT-011, MK-3475, MPDL3280A, CP-870,893, TRX518, or TRX385.

9. The immunotherapeutic composition of claim 1, wherein said immunostimulatory therapeutic nucleic acid molecule(s) is capable of expressing one or more alloantigen(s) that stimulate T-cell immunity against a tissue or a cell.

10. The immunotherapeutic composition of claim 9, wherein said alloantigen(s) comprise a class I major histocompatibility complex (MHC) antigen, a β2 microglobulin, or a cytokine.

11. The immunotherapeutic composition of claim 10, wherein said class I major histocompatibility complex (MHC) antigens are HLA-B7 and/or β2-microglobulins.

12. The immunotherapeutic composition of claim 11, wherein the binding component(s) is a monoclonal antibody that binds with specificity to CTLA-4.

13. The immunotherapeutic composition of claim 1, wherein said pharmaceutically acceptable carrier is a cationic lipid-based system.

14. The immunotherapeutic composition of claim 13, wherein the cationic lipid-based system is 1,2-dimyristyloxypropyyl-3-dimethylhydroxyetheyl ammonium bromide (DMRIE) with dioleoyl phosphatidylethanolamine (DOPE).

15. A method for treating or preventing a cancer in a mammal comprising administering a pharmaceutically effective amount of a composition comprising an antibody having specificity to CTLA-4, PD-1, PD-L1, PD-L2, CD40, OX40, CD137, GITR, ILT2, or ILT3 or an antibody having specificity to a ligand of CTLA-4, PD-1, PD-L1, PD-L2, CD40, OX40, CD137, GITR, ILT2, or ILT3, and an immunostimulatory therapeutic nucleic acid that expresses HLA-B7 and β2-microglobulins to a mammal.

16. The method of claim 15, wherein the mammal is a human.

17. The method of claim 15, wherein the cancer is selected from the group consisting of melanoma, squamous cell carcinoma, basal cell carcinoma, breast cancer, head and neck carcinoma, thyroid carcinoma, soft tissue sarcoma, bone sarcoma, testicular cancer, prostatic cancer, ovarian cancer, bladder cancer, skin cancer, brain cancer, angiosarcoma, hemangiosarcoma, mast cell tumor, primary hepatic cancer, lung cancer, pancreatic cancer, gastrointestinal cancer, renal cell carcinoma, hematopoietic neoplasia, and metastatic cancer thereof.

18. The method of claim 17, wherein the cancer is melanoma, squamous cell carcinoma, or basal cell carcinoma.

19. The method of claim 18, wherein the cancer is melanoma.

20. A kit comprising a first container comprising a controlled release formulation of an antibody selected from the group consisting of ipilimumab, BMS-936558, BMS-936559, BMS-663513, CT-011, MK-3475, MPDL3280A, CP-870,893, TRX518, or TRX385, said formulation comprising an amount of antibody effective to treat or reduce and/or prevent melanoma, and a second container comprising an immunostimulatory therapeutic nucleic acid molecule and a pharmaceutically acceptable carrier.

21. The kit of claim 20, wherein the immunostimulatory therapeutic nucleic acid molecule and pharmaceutically acceptable carrier comprise a controlled/sustained/extended/prolonged release formulation of a plasmid encoding HLA-B7 heavy chain and β2-microglobulin, formulated with DMRIE-DOPE in an amount effective to treat or reduce and/or prevent melanoma.

22. The kit of claim 21, further comprising a puncture needle or catheter.

23. The kit of claim 21, further comprising a package insert.
